(19) United States
(12) Patent Application Publication
(10) Pub. No.: US 2023/0322661 A1
(43) Pub. Date: Oct. 12, 2023

(54) Synthesis of N,N-Branched Sulfamoyl Fluoride Compounds Using Bismuth Trifluoride

(71) Applicant: SES Holdings Pte. Ltd., Singapore (SG)

(72) Inventors: Rajendra P. Singh, Woburn, MA (US); Qichao Hu, Arlington, MA (US); Johnathan Sheiman, Weston, MA (US)

(21) Appl. No.: 18/021,751

(22) PCT Filed: Aug. 19, 2021
(86) PCT No.: PCT/IB2021/057654

§ 371 Date: Feb. 16, 2023

Related U.S. Application Data
(60) Provisional application No. 63/068,495, filed on Aug. 21, 2020.

(51) Int. Cl. C07C 303/22

(52) U.S. Cl. CPC C07C 303/22

(57) ABSTRACT

Methods of producing N,N-branched sulfamoyl fluoride compounds of the formula F-S(O)₂-NR₂ by contacting bismuth trifluoride with an N,N-branched sulfamoyl nonfluorohalide compound of the formula X-SO₂NR₂, wherein X=chlorine (Cl), bromine (Br), or iodine (I), and each R is, independently, a linear or branched alkyl, fluoroalkyl, alkenyl, fluoroalkenyl, alkynyl, or fluoroalkynyl with 1 to 12 carbon atoms, to fluorinate the N,N-branched sulfamoyl nonfluorohalide compound. This is a non-aqueous method, the purity of product is very high, and the desired product can be isolated in quantitative yield. The N,N-branched sulfamoyl fluoride compounds so produced are useful in various applications including as electrolyte solvents and additives in electrochemical devices, such as lithium batteries and capacitors, and in biological fields, among others.

### RELATED APPLICATION DATA

This application claims the benefit of priority of U.S. Provisional Patent Application Ser. No. 63/068,495, filed on Aug. 21, 2020, and titled “SYNTHESIS OF N,N-DIALKYL SULFAMOYL FLUORIDE”, the content of which is incorporated herein by reference in its entirety.

### FIELD OF THE DISCLOSURE

The present disclosure relates to methods of producing branched sulfamoyl fluoride compounds. More particularly, the present disclosure is directed to methods of producing N,N-branched sulfamoyl fluoride compounds using bismuth trifluoride.

### BACKGROUND

Incorporation of fluorine into molecules often results in a significant change in the physical and chemical properties of the molecules. Some fluorine-containing compounds have high electrochemical stability and are useful in electrochemical energy storage devices such as batteries and electric double layer capacitors and in fields of biology.

The compound N-(fluorosulfonyl) dimethylamine (FSO₂NMe₂) has been proposed as a solvent or additive for lithium-ion batteries (Chinese Patent No. CN 1 289 765A). At present, FSO₂NMe₂ is not commercially available in large amounts.

FSO₂NMe₂ was first prepared in the 1930s by metathesis between N-chlorosulfonyl dimethylamine (CISO₂NMe₂) and potassium, sodium, or zinc fluoride in water (French Patent No. FR 806 383; German Patent No. DE 667 544; U.S. Pat. No. 2,130,038). This was an aqueous method, and yield was low.

FSO₂NMe₂ has also been prepared by the reaction of CISO₂NMe₂ with antimony trifluoride (SbF₃) in the presence of antimony pentafluoride (SbF₅) (Heap, R., Saunders, B. C., Journal of the Chemical Society (Resumed), 1948, 1313-1316), and by the reaction of CISO₂NMe₂ with anhydrous hydrogen fluoride (HF) at 80° C.-90° C. (German Patent No. DE 1 943 233 (1971)). The obtained product, contaminated with chloride, is not suitable for use in lithium batteries.

FSO₂NMe₂ has also been prepared by the reaction of N,N-dimethylaminosulfamide (Me₂NSO₂NH₂) with fluorosulfonyl isocyanate (FSO₂N=C=O) at 80° C. (Appel, R.; Montenarh, M., Chemische Berichte, 1977, 110, 2368-2373). Various byproducts were detected in the synthesis.

Generally, there are four known examples of the reaction of sulfuryl fluoride (SO₂F₂) with secondary amines as described below. The four known reactions employ cryogens (or catalysts).
1. The reaction of SO₂F₂ with a secondary amine was first performed in 1948 (Emeléus, H. J., Wood, J. F., Journal of the Chemical Society (Resumed), 1948, 2183-2188). In this paper, diethylamine (Et₂NH) was dropped into a cooled (−78° C.) solution of SO₂F₂ in ethyl ether, and the product, FSO₂NEt₂, was obtained in a yield of 35%.
2. The reaction of SO₂F₂ with piperidine (HN(CH₂)₅) was performed in 1982 (Padma, D. K., Subrahmanya Bhat, V., Vasudeva Murthy, A. R., Journal of Fluorine Chemistry, 1982, 20, 425-437). SO₂F₂ was added into piperidine in ether at −196° C., followed by warming. Either FSO₂N(CH₂)₅ or SO₂(N(CH₂)₅)₂ was obtained, depending on the amount of piperidine used. Separation of the desired compound from byproducts proved difficult.
3. The reaction of two more secondary amines with SO₂F₂, under ambient conditions, is described in a patent application by Dong and Sharpless (International Patent Application Publication No. WO 2015/188120). In this published application, diallyl amine and dipropargyl amine react with SO₂F₂ in the presence of an equivalent of an activating agent in a solvent. The solvents, such as tetrahydrofuran (THF) and dichloromethane, were particularly described. Dong and Sharpless assert in the published application that “activated amines can even react in buffer at pH 8” but gave no examples of activated amines and did not further elaborate.
4. Synthesis of DSF is also reported by reaction of dimethylamine with gaseous sulfuryl fluoride. In this process, mixtures of products such as Me₂SO₂F+Me₂NH₂F+SO₂(NMe₂)₂ are formed and are hard to separate from the desired product. Due to these drawbacks, the scale up of DSF is not economical.

Accordingly, there is a need for a relatively safer, economical, and high yield method for producing high-purity N,N-dialkyl sulfamoyl fluoride compounds, such as high-purity N,N-dimethyl sulfamoyl fluoride and derivatives thereof, especially at commercial scale.

### SUMMARY

In an implementation, the present disclosure is directed to a method for producing an N,N-branched sulfamoyl fluoride compound of the formula F-SO2-NR2. The method includes contacting an N,N-branched sulfamoyl nonfluorohalide compound of the formula X-SO2-NR2 with BiF3 under conditions sufficient to produce a mixture containing the sulfamoyl fluoride compound F-SO2-NR2 and BiX3 as a byproduct; wherein X=Cl, Br, or I and each R is, independently, a linear, branched, or cyclic alkyl, fluroalkyl, alkenyl, fluoroalkynyl, alkynyl or fluoroalkynyl.

### DETAILED DESCRIPTION

In some aspects, the present disclosure is directed to methods of producing N,N-dimethyl sulfamoyl fluoride (DMSF) and derivatives thereof, or, more generally N,N-branched sulfamoyl fluoride compounds, of the formula F-S(O)₂-NR₂ (I) by contacting bismuth trifluoride (BiF₃) with an N,N-branched sulfamoyl nonfluorohalide compound of the formula X-SO₂NR₂ (II), wherein X=chlorine (Cl), bromine (Br), or iodine (I), and each R is, independently, a linear or branched alkyl, fluoroalkyl, alkenyl, fluoroalkenyl, alkynyl, or fluoroalkynyl with 1 to 12 carbon atoms, to fluorinate the N,N-branched sulfamoyl nonfluorohalide compound. This is a non-aqueous method, the purity of product is very high, and the desired product can be isolated in quantitative yield. The N,N-branched sulfamoyl fluoride compounds so produced are useful in various applications including as electrolyte solvents and additives in electrochemical devices, such as lithium batteries and capacitors, and in biological fields, among others.

In an example, the fluorination reaction of an N,N-branched sulfamoyl chloride using BiF₃ is as follows:

Cl—SO₂—NR₂+⅓BiF₃→F—SO₂—NR₂+⅓BiCl₃

In some embodiments, the BiX₃ that is produced as a byproduct of the reaction can be converted back to BiF₃. For example, BiCl₃, which is produced as a byproduct when X in Formula II is Cl, can be converted back to BiF₃ via reacting it with NaOH to isolate Bi₂O₃, followed by treatment with aqueous hydrogen fluoride (HF). Further examples of BiX₃ regeneration are discussed below.

Definitions

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Alkyl groups can be optionally substituted with an alkoxide (i.e., —OR^{a}, where R^{a} is alkyl) and/or other functional group(s) that are either protected or non-reactive under a given reaction condition. Exemplary alkyl groups include, but are not limited to, methyl, ethyl, n-propyl, 2-propyl, tert-butyl, pentyl, and the like.

“Alkenyl” means a linear monovalent hydrocarbon moiety of two to twelve, typically two to six, carbon atoms or a branched monovalent hydrocarbon moiety of three to twelve, typically three to six carbon atoms, containing at least one carbon-carbon double bond. Alkenyl groups can optionally be substituted with one or more functional groups that are either protected or non-reactive under a given reaction condition. Exemplary alkenyl groups include, but are not limited to, vinyl, propenyl, butenyl, and the like.

“Alkynyl” means a linear monovalent hydrocarbon moiety of two to twelve, typically two to six carbon atoms, or a branched monovalent hydrocarbon moiety of three to twelve, typically three to six carbon atoms, containing at least one carbon-carbon triple bond. Alkynyl groups can optionally be substituted with one or more functional groups that are either protected or non-reactive under a given reaction condition. Exemplary alkynyl groups include, but are not limited to, ethynyl, propynyl, butynyl, and the like.

“Cycloalkyl” refers to a non-aromatic, saturated, monovalent mono- or bi-cyclic hydrocarbon moiety of three to ten ring carbons. The cycloalkyl can be optionally substituted with one, two or three substituents within the ring structure that are either protected or unreactive under a given reaction condition.

“Cycloalkenyl” refers to a non-aromatic, monovalent mono- or bi-cyclic hydrocarbon moiety of three to ten ring carbons having at least one carbon-carbon double bond within the ring system. The cycloalkyl can be optionally substituted with one, two or three substituents within the ring structure that are either protected or unreactive under a given reaction condition.

As used herein and in the appended claims, the terms “treating”, “contacting”, and “reacting” refer to adding or mixing two or more reagents under appropriate conditions to produce the indicated and/or the desired product. It should be appreciated that the reaction that produces the indicated product and/or the desired product may not necessarily result directly from the combination of two reagents initially added; i.e., there may be one or more intermediates produced in the mixture that ultimately lead(s) to desired product.

As used herein and in the appended claims, the term “anhydrous” refers to having about 1% by weight of water or less, typically about 0.5% by weight of water or less, often about 0.1% by weight of water or less, more often about 0.01% by weight of water or less, and most often about 0.001% by weight of water or less. Within this definition, the term “substantially anhydrous” refers to having about 0.1% by weight of water or less, typically about 0.01% by weight of water or less, and often about 0.001% by weight of water or less.

Throughout the present disclosure, the term “about” when used with a corresponding numeric value refers to ±20% of the numeric value, typically ±10% of the numeric value, often ±5% of the numeric value, and most often ±2% of the numeric value. In some embodiments, the term “about” can mean the numeric value itself.

Illustrative Methods

As noted above, some aspects of this disclosure are directed toward overcoming one or more of the problems discussed in the Background section above associated with producing an N,N-branched sulfamoyl fluoride as discussed above. In some embodiments, methods of this disclosure use bismuth trifluoride as a fluorinating reagent. In some embodiments, methods of this disclosure allow for the used bismuth reagent to be recycled to regenerate bismuth trifluoride.

One aspect of the present disclosure provides a method for producing an N,N-branched sulfamoyl fluoride compound of the formula

F—SO₂—NR₂ (I)

by contacting an N,N-branched sulfamoyl nonfluorohalide compound of the formula

X—SO₂—NR₂

with BiF₃ under conditions sufficient to produce said fluorinated compound of Formula I. Typically, such methods also produce BiX₃ as a byproduct. In compounds of Formulas I and II, X and each R can be as defined above.

Such methods typically comprise contacting the N,N-branched sulfamoyl nonfluorohalide compound (II) with BiF₃ under conditions sufficient to produce said fluorinated compound (I) and BiX₃ as a byproduct. BiX₃ byproduct can be recycled to regenerate BiF₃ as described above and/or in the relevant literature, such as in the references incorporated herein by reference above.

The conditions sufficient for producing the fluorinated compound (I) can be quite broad. For example, the temperature may be about 0° C. to about 50° C., about 20° C. to about 70° C., about 20° C. to about 90° C., and about 20° C. to about 110° C. Depending upon the initial temperatures of the reactants and/or the reaction vessel, reaching the proper conditions may require heating or cooling the mixture of the reactants in the reaction vessel. However, other temperatures and pressures, including pressures above atmospheric pressure or below atmospheric pressure, may be used in conjunction with temperatures and reaction times that yield satisfactory results.

In some embodiments, the reaction time may be about 0.1 hr to about 24 hrs, or more. It is noted that temperature and time can have a big impact on the yield. For example, good results (e.g., >93% yield) can be achieved at 110° C. over 15 hrs at atmospheric pressure. However, the reaction can occur at room temperature, but the yield can be <5% in 24 hrs. In some embodiments, mixing (e.g., by stirring) is desirable to ensure that the reaction is complete as possible throughout the mixture. Consequently, heating and proper mixing is desirable to achieve complete reaction and higher yields.

In some embodiments, for example, embodiments involving large-scale production (e.g., production starting with N,N-branched sulfamoyl nonfluoride in an amount greater than 20 g, greater than 100 g, greater than 200 g, greater than 500 g, or greater than 1000 g), it can be beneficial to heat the mixture in two or more stages at differing temperatures to minimize or eliminate decomposition products that would contaminate the desired reaction product. For example, heating the mixture in large-scale production to too high a temperature too quickly can cause the reaction, which is exothermic, to proceed too quickly and thereby produce an excessive amount of heat that can result in unwanted decomposition products being formed in the mixture that contaminate the desired reaction product. When staged heating is used, in some embodiments the heating of the mixture starts with initially applying heat of a relatively lower temperature, followed by raising the temperature of the applied heat, for example, either incrementally or gradually. When incremental heating is used, the initial lower temperature may be held for a first amount of time after which the temperature is raised to at least one higher temperature, with each higher temperature being held for a desired amount of time. In some embodiments, the amount of time that the initial relatively lower temperature is held can be shorter than the total amount of time that one or more relatively higher temperatures is held.

From experimentation it has been found that applying moderate heat (e.g., heating at a temperature less than about 80° C. and starting with a temperature less than about 65° C.) while the bulk of the high-heat-producing exothermic reaction is occurring can yield good result, i.e., minimize unwanted decomposition byproducts. Generally, the length of time that the temperature should be maintained below about 80° C. will vary with the starting amounts of the reactants and the temperature(s) used. Experimentation has also shown that staging increases in temperature in increments of less than about 15° C. or about 10° C. or less and using three or more heating stages over a period of about three or more hours can yield particularly good results for starting amounts of N,N-branched sulfamoyl nonfluoride of about 1000 g to 2000 g or more. In an example, excellent results were achieved using the following staged heating scheme when starting with 2000 g of N,N-branched sulfamoyl nonfluoride: 50° C. for 1 hour, 60° C. for 1 hour, 70° C. for 1 hour, 80° C. for 1 hour, 90° C. for 1 hour, and >90° C. (e.g., 100° C. to 110° C.) for a suitable additional amount of time.

In some embodiments, the yield is typically in a range of about 70% to about 99%, greater than about 80%, greater than about 90%, greater than about 95%, or greater than about 98%. In some embodiments, the purity of the desired product of Formula I is typically in a range of about 90% to about 99.99%. In one example, DMSF has been isolated at >99.8% based on ¹⁹F and ¹H nuclear magnetic resonance spectroscopy (NMR). In some embodiments, one or more distillations and crystallizations may be needed to achieve the highest purity N,N-branched sulfamoyl fluoride product.

The anhydrous nature of the synthesis of the present disclosure can allow the purity to be greater than 99%. In contrast, most of the know DMSF synthesis processes have significant byproducts and have high water content that need to be removed before using the DMSF, for example, in lithium-metal batteries. The process of the present disclosure is very clean. In some embodiments, only a second distillation may be needed to remove any halide impurities. The present process is anhydrous and free of ionic halide impurities. In some embodiments, a 2% to 3% molecular sieve is used to remove water. In one example, after molecular-sieve drying, the water content of the DMSF is <5 ppm. This low level of water content has been observed kilogram-order size synthesis.

In an example involving DMSF, at typical reaction conditions the DMSF is a liquid and the bismuth trichloride is solid. With these states, fairly simple large scale and continuous processes can be implemented. For example, various combinations of filtering, distillation, and other separating techniques can be used to separate the liquid DMSF and the solid bismuth trichloride. As an example, the mixture of DMSF and BiCl₃ may be treated with an inert organic solvent comprising at least one alkane, such as hexane, a chloroalkane (e.g., dichloromethane), and/or a fluoroalkane, among others, followed by distillation. As noted above, the liquid DMSF can be further distilled to remove halide impurities and/or molecular-sieve dried to remove unwanted water. In some embodiments, the above reaction may be performed as a continuous reaction where both reactants can be brought in contact at 100° C. to 150° C. to keep the DMSF in a liquid state and the bismuth trichloride in a solid state. It is noted that while DMSF is the desired reaction product in this example, the reaction conditions, separation techniques (including use of solvent), filtering, and other aspects can also be applied to non-DMSF desired N,N-branched sulfamoyl fluoride synthesis products. An example and non-exhaustive set of other N,N-branched sulfamoyl fluoride products that can be synthesized using methods of the present disclosure includes N,N-diethyl sulfamoyl fluoride, N-ethyl-N-methyl sulfamoyl fluoride, and N-ethyl-N-methoxyethyl sulfamoyl fluoride, to name a few.

It is noted that the foregoing reaction takes place in the absence of a solvent. However, in some cases it may be desirable to include a solvent in the reaction mixture. Example solvents that could be included in the reaction mixture, either singly or in any combination, include, but are not limited to, alkanes, ethers, halocarbons, and aromatic solvents.

BiF₃ Regeneration:

One of the advantages of methods of the present disclosure is regeneration of bismuth(III) oxide, (Bi₂O₃) from bismuth trichloride (BiCl₃) that is formed in the reaction when X in Formula II is chlorine. Typically, bismuth trichloride can be converted to bismuth(III) oxide by treating with sodium carbonate in water at 90° C. for 10 minutes. Water insoluble bismuth(III) oxide can be obtained by filtration and washing with water to remove sodium chloride. The isolated bismuth(III) oxide can be reacted with either anhydrous HF or aqueous HF to regenerate bismuth trifluoride. Typically, bismuth(III) oxide can be taken in a polytetrafluoroethylene (PTFE) vessel and treated with excess aqueous HF until all the solids reacted. BiF₃ is insoluble in water and can be isolated by filtration and drying in vacuum, for example, at a temperature in a range of 60° C. to 100° C.

Example techniques that can be used for regenerating BiF₃ from BiCl₃ may be found, for example, in U.S. 8,377,406 B1, titled “Synthesis of bis(fluorosulfonyl)imide”, issued Feb. 19, 2013, in the names of Rajendra P. Singh, Jerry Lynn Martin, and Joseph Carl Poshusta, and in Greenwood, Norman N.; Earnshaw, Alan (1997), Chemistry of the Elements (2nd ed.) Butterworth-Heinemann, ISBN 978-0-08-037941-8.). Each of these references is incorporated by reference herein for its teachings relating to regenerating BiF₃ from BiCl_{3.}

Other processes of regenerating BiF₃ from BiCl₃ may be used. For example, the BiCl₃ can be treated with anhydrous HF to produce BiF₃ and HCl as a byproduct as follows:

BiCl₃+3HF=BiF₃+3HCl

Additional objects, advantages, and novel features of this disclosure will become apparent to those skilled in the art upon examination of the following examples thereof, which are not intended to be limiting. In the examples, procedures that are constructively reduced to practice are described in the present tense, and procedures that have been carried out in the laboratory are set in the past tense.

EXAMPLES

Unless otherwise stated, all chemicals used were of reagent grade. N,N-dimethyl sulfamoyl chloride and bismuth trifluoride used herein were anhydrous. All laboratory-scale reaction operations should be carried out in a well-ventilated fume hood, and proper personal-protection equipment (PPE), such as a lab coat, safety glasses, and gloves, should be worn.

Example 1: Synthesis of N,N-dimethyl sulfamoyl fluoride:

Bismuth trifluoride, BiF₃, (2000 g, 7.52 mole) was weighed in a 3 L round-bottom flask, and N,N-dimethyl sulfamoyl chloride (2000 g, 13.93 mole) was added to the flask at room temperature. The flask was attached to a dry nitrogen or argon line, followed by connecting to a mechanical stirrer. The reaction mixture was heated with an oil bath to 65° C. for 2 hrs, with stirring followed by heating to 100° C.-110° C. for an additional 15 hrs. The reaction mixture was cooled and then distilled at reduced pressure (50° C./20 mmHg) to produce N,N-dimethyl sulfamoyl fluoride in >95% yield as a clear colorless liquid. The identity of the product was confirmed by ¹⁹F and ¹H nuclear magnetic resonance spectroscopy (NMR). The reaction of this example is illustrated immediately below.

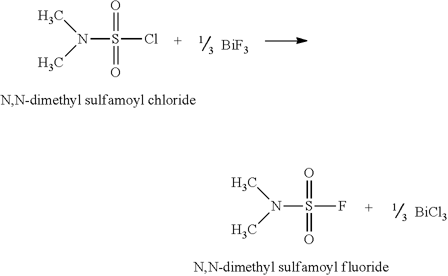

Example 2: Synthesis of N,N-dimethyl sulfamoyl fluoride:

Bismuth trifluoride, BiF₃, (25 g, 0.094 mole) was weighed in a 100 mL round-bottom flask, and N,N-dimethyl sulfamoyl chloride (25 g, 0.174 mole) was added to the flask at room temperature. The flask was attached to a dry nitrogen or argon line, followed by connecting to a mechanical stirrer. The reaction mixture was heated with an oil bath to 100° C.-110° C. for 15 hrs.

The reaction mixture was cooled, and 20 g anhydrous dichloromethane was added, and the mixture was stirred well and then filtered to separate solid BiCl₃. Dichloromethane from the filtrate was removed by normal distillation, and the desired product was distilled at reduced pressure (50° C./20 mmHg) to provide N,N-dimethyl sulfamoyl fluoride in 94% yield as a clear colorless liquid. The identity of the product was confirmed by ¹⁹F and ¹H NMR. The reaction of this example is the same as illustrated immediately above.

Example 3: Synthesis of N,N-diethyl sulfamoyl fluoride:

Bismuth trifluoride, BiF₃, (20.8 g, 0.078 mole) was weighed in a 100 mL round-bottom flask, and N,N-diethyl sulfamoyl chloride (25.84.1 g, 0.145 mole) was added. The flask was attached to a dry nitrogen or argon line, followed by connecting to a mechanical stirrer. The reaction mixture was heated with an oil bath to 65° C. for 2 hrs, with stirring followed by heating to 100-110° C. for an additional 15 hrs. The reaction mixture was cooled and distilled at reduced pressure to give N,N-diethyl sulfamoyl fluoride in >93% yield as a clear colorless liquid. The reaction of this example is illustrated immediately below.

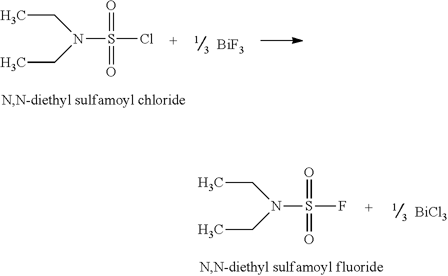

Example 4: Synthesis of N-methyl-N-ethyl sulfamoyl fluoride:

Bismuth trifluoride, BiF₃, (22.57 g, 0.084 mole) was weighed in a 250 mL round-bottom flask, and N-methyl-N-ethyl sulfamoyl chloride (24.88 g, 0.1581 mole) was added. The flask was attached to a dry nitrogen or argon line, followed by connecting to a mechanical stirrer. The reaction mixture was heated with an oil bath to 70° C. for 2 hrs, with stirring followed by heating to 110° C. for an additional 15 hrs. The reaction mixture was cooled and distilled at reduced pressure to give N-methyl-N-ethyl sulfamoyl fluoride in >94% yield as a clear colorless liquid. The reaction of this example is illustrated immediately below.

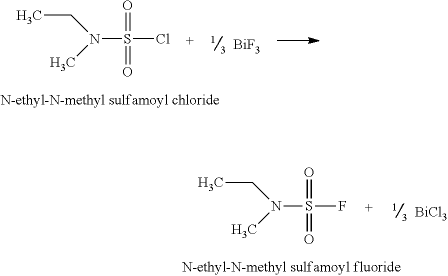

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method for producing an N,N-branched sulfamoyl fluoride compound of the formula F-SO₂-NR₂, the method comprising:
contacting an N,N-branched sulfamoyl nonfluorohalide compound of the formula X-SO₂-NR₂ with BiF₃ under conditions sufficient to produce a mixture containing the sulfamoyl fluoride compound F-SO₂-NR₂ and BiX₃ as a byproduct;
wherein X=Cl, Br, or I and each R is, independently, a linear, branched, or cyclic alkyl, fluroalkyl, alkenyl, fluoroalkynyl, alkynyl or fluoroalkynyl.

2. The method of claim 1, wherein the conditions sufficient to produce the N,N-branched sulfamoyl fluoride compound and BiX₃ as a byproduct include heating and mixing the mixture.

3. The method of claim 2, wherein the heating includes heating the mixture to a temperature in a range of 20° C. to 200° C.

4. The method of claim 3, wherein the heating includes heating the mixture to an initial temperature followed by heating the mixture to a second temperature higher than the initial temperature.

5. The method of claim 4, wherein the initial temperature is less than 80° C. and the second temperature is equal to or greater than 100° C.

6. The method of claim 3, wherein the method further includes separating the N,N-branched sulfamoyl fluoride compound from the mixture by distillation.

7. The method of claim 3, further comprising treating the mixture with inert organic solvent, followed by removing the BiX3 from the mixture by filtration and separating the N,N-branched fluoride compound from the mixture by distillation.

8. The method of claim 6, wherein the inert organic solvent comprises at least one alkane.

9. The method of claim 7, wherein the at least one alkane is a chloro- or fluoro-alkane.

10. The method of claim 2, wherein the contacting and heating are performed at atmospheric pressure, and the method further comprises separating the N,N-branched sulfamoyl fluoride compound from the mixture by distillation.

11. The method of claim 1, wherein X is Cl.

12. The method of claim 1, wherein Xis Br.

13. The method of claim 1, wherein Xi is I.

14. The method of claim 1, wherein each R is selected from the group consisting of —CH₃, —CH₂CH₃, and —CH₂CH₂OCH_{3.}

15. The method of claim 14, wherein the N,N-branched sulfamoyl fluoride compound is selected from the group consisting of N,N-dimethyl sulfamoyl fluoride, N,N-diethyl sulfamoyl fluoride, N-ethyl-N-methyl sulfamoyl fluoride, and N-ethyl-N-methoxyethyl sulfamoyl fluoride.

16. The method of claim 1, wherein the method further includes separating the N,N-branched sulfamoyl fluoride compound from the mixture by distillation.

17. The method of claim 1, further comprising treating the mixture with a solvent, followed by removing the BiX3 from the mixture by filtration and separating the N,N-branched fluoride compound from the mixture by distillation.

18. The method of claim 17, wherein the solvent comprises anhydrous hexane or dichloromethane.

19. The method of claim 1, wherein the contacting is performed at atmospheric pressure, and the method further comprises separating the N,N-branched sulfamoyl fluoride compound F-SO₂-NR₂ from the mixture by distillation.

20. The method of claim 1, wherein the yield of the N,N-branched sulfamoyl fluoride compound is at least 70%.

21. The method of claim 1, wherein the yield of the N,N-branched sulfamoyl fluoride compound is at least 90%.
